(19) United States
(12) Patent Application Publication
(10) Pub. No.: US 2026/0204402 A1
(43) Pub. Date: Jul. 16, 2026

(54) METHOD FOR MANAGING MEDICAL PRODUCT USAGE IN A HEALTHCARE ENVIRONMENT

(71) Applicant: Mölnlycke Health Care AB, Mölndal (SE)

(72) Inventors: Amy Duong, Lawrenceville, GA (US); Joe M. Rivera, Moorestown, NJ (US); Sarah L. Rodgers, Alpharetta, GA (US); Jonathan L. Schopp, Broussard, LA (US); Sanjay Mistry, Johns Creek, GA (US)

(21) Appl. No.: 19/016,185

(22) Filed: Jan. 10, 2025

(51) Int. Cl. G16H 40/40

(52) U.S. Cl. CPC G16H 40/40

(57) ABSTRACT

The present disclosure relates to a computer-implemented method, system, and corresponding computer program product for managing medical product usage in a healthcare environment. The disclosed solution involves receiving input at an electronic user device identifying a medical product selected for use on a patient and collecting data from one or more input sources associated with the electronic user device. A product usage compliance level is determined based on the collected data, either using the electronic user device or a server. The system evaluates the accessibility of the selected medical product within the healthcare unit and generates a recommendation for an action based on a combination of the determined compliance level and the accessibility of the medical product. The recommendation is provided to a healthcare professional, enabling proactive management of inventory and application accuracy to improve patient care and reduce risks associated with improper medical product usage.

### TECHNICAL FIELD

The present disclosure relates to a computer-implemented method, system, and corresponding computer program product for managing medical product usage in a healthcare environment.

### BACKGROUND

The selection of appropriate medical products, such as wound care products, is a critical component of ensuring patient safety and improving overall care quality. Choosing the most suitable product helps address immediate care needs while supporting long-term healing outcomes. Such considerations are particularly important in dynamic healthcare environments, where patient requirements can vary significantly depending on factors such as risk of pressure ulcers, mobility, and skin condition.

US20210391065A1, for example, describes a system for selecting an optimal medical product in the form of a wound product for a patient by analyzing wound-specific parameters such as size, exudate level, and healing trajectory. The disclosed system matches wound products with desired functional properties, enabling personalized product recommendations that aim to improve treatment outcomes by tailoring selections to the unique needs of the patient and their wound.

Among the medical products available, and thus selectable, prophylactic medical products, such as prophylactic wound dressings are among the most essential tools for reducing the risk of pressure ulcers, preventing skin breakdown, and improving overall patient outcomes. Proper use of such medical products plays a vital role in mitigating pressure-related injuries and promoting optimal healing conditions. To maintain high standards of patient care and minimize preventable complications, it is crucial that these products are consistently and effectively utilized.

While such advancements mark significant progress in product availability, further improvements are needed to ensure that appropriate medical products are consistently used in clinical practice. Advanced technologies capable of leveraging data processing and trend analysis over time could provide healthcare professionals with more precise assessments of patient-specific needs. Such technologies could potentially facilitate the proactive management of medical products, enhance decision-making for healthcare personnel, and ultimately improve patient outcomes.

### SUMMARY

In view of the above-mentioned and other drawbacks of the prior art, it is an object of the present disclosure to provide improvements in how to manage medical product usage in a healthcare environment.

According to an aspect of the present disclosure, it is therefore provided a method for managing medical product usage at a healthcare unit, comprising receiving, at an electronic user device, input identifying a medical product selected for use in relation to a patient, determining, using the electronic user device and based on data obtained from one or more input sources associated with the electronic user device, a product usage compliance level for the selected medical product applied, generating, by a server arranged in communication with the electronic user device, a recommendation for an action based on a combination of an accessibility of the selected medical product within the healthcare unit and the determined product usage compliance level, and providing, by the server, the recommendation to a healthcare professional associated with the healthcare unit.

The present disclosure enables the proactive management of medical products by addressing two critical aspects, namely by ensuring the consistent availability of the medical products and verifying that the selected medical products are correctly used in relation to the patient. Accordingly, by determining a medical product usage compliance level based on data collected at the point of care and combining this with an assessment of the accessibility of the medical product within the healthcare unit, the disclosed method generates actionable recommendations tailored to address deficiencies, possibly in both areas.

Such an approach ensures that healthcare professionals receive timely and accurate guidance, enabling them to address issues such as insufficient inventory or improper product usage before they negatively impact patient outcomes. By focusing on both usage compliance and product availability, the present disclosure overcomes limitations in existing systems, which may emphasize product selection without addressing the challenges of their consistent and effective use in practice.

The core idea is to employ a distributed system involving an electronic user device and a server to dynamically collect, analyze, and process relevant data for managing medical product usage. Such an implementation allows for real-time recommendations that support healthcare personnel in maintaining high standards of care while minimizing disruptions to clinical workflows.

An advantage of the present disclosure is its ability to integrate insights from multiple data sources into a single recommendation process, as such simultaneously addressing gaps in compliance and availability of the selected medical product. Additionally, the solution according to the present disclosure supports healthcare professionals by identifying areas where further actions, such as restocking or training, may be necessary, with the general intention to improve the overall quality of care provided to patients.

It should be understood that the scheme according to the present disclosure may be applied simultaneously to multiple patients and multiple types of medical products, whereby aggregated data may be generated by the server. The feedback from the server may be provided following the assessment of the healthcare unit, or directly in real time as the assessment progresses.

Preferably, the medical product is selected from a group comprising, for example, medical dressings, turning and positioning devices, and pressure ulcer prevention devices. The medical products may include prophylactic dressings such as sacral or heel dressings for wound prevention and treatment, as well as fluidized positioners or patient handling systems designed to support patient turning and positioning. Examples of suitable medical products include but are not limited to Mölnlycke Mepilex Border Sacrum dressings, Mölnlycke Mepilex Border Heel dressings, Mölnlycke Mepilex Border Flex dressings, Mölnlycke Mepilex Border Lite dressings, Mölnlycke Z-Flex Fluidized Heel Boots for pressure ulcer prevention, Mölnlycke Tortoise Turning and Positioning Systems for patient handling, and Mölnlycke Z-Flo Fluidized Positioners, including neonatal care devices. Other similar medical products, by any manufacturer, are to be considered and are within the scope of the present disclosure.

In one embodiment of the present disclosure, the determining the product usage compliance level comprises presenting, at a graphical user interface (GUI) configured at the electronic user device, a set of product usage-related questions, and receiving, at the GUI of the electronic user device, responses from a user, wherein the responses are used to determine the product usage compliance level. In line with the present embodiment, the GUI is preferably designed to display a structured set of questions related to the usage of the medical product, such as whether the medical product is fully adhered, aligned correctly, or applied to the intended site, etc. The compliance of course depends on the type of medical product used in relation to the patient. Responses provided by the user are captured via the same interface and processed to derive the compliance level. Such an implementation leverages user input as a primary data source, which may be supplemented by visual or sensor-based inputs in other embodiments.

Accordingly, the solution according to the present disclosure ensures that key application-related parameters are actively assessed, even in situations where automated data collection methods (e.g., image analysis) are unavailable or insufficient. The interaction through the GUI standardizes how compliance is evaluated across different users, reducing variability in assessments and ensuring critical application aspects are addressed systematically, enhancing the overall reliability of compliance determination and enables effective decision-making by healthcare personnel.

An advantage following the present embodiment is the possibility of enabling personnel with varying levels of expertise to actively participate in the compliance assessment process without requiring specialized tools. Furthermore, such a solution promotes flexibility, as the GUI can be adapted to present different questions based on the type of medical product or specific clinical requirements. Additionally, the suggested approach provides a cost-effective and scalable method for evaluating compliance, particularly in settings where advanced sensor or imaging systems may not be feasible. As such, the combination of user input and automated processes ensures a comprehensive assessment of product usage compliance.

In an alternative embodiment, determining the product usage compliance level comprises capturing, using an image device associated with the electronic user device, an image of the used medical product and analyzing, using the server, the image to determine the product usage compliance level. As such, the product usage compliance level may in such an embodiment be determined by leveraging image-based analysis. An image device, such as a built-in or external camera associated with the electronic user device, is used to capture an image of the used medical product. The image is then transmitted to the server, where it undergoes analysis to evaluate parameters such as medical product alignment, coverage, adherence, and condition. By relying on image processing techniques, such an implementation minimizes the reliance on subjective user input, instead deriving compliance metrics through automated, standardized analysis, typically performed by the server.

The integration of image capture and analysis achieves an improvement in the precision and objectivity of compliance determination. The image data provides a detailed visual representation of the usage of the medical product, allowing the server to detect even subtle deviations or errors that may not be directly apparent through manual inspection. The processing capabilities of the server enable consistent evaluation criteria to be applied across all assessments, reducing variability and enhancing the accuracy of compliance determination.

An advantage of such an embodiment is its ability to provide healthcare personnel with a highly accurate and consistent assessment of product usage compliance, regardless of their level of expertise. The use of image-based analysis reduces the potential for human error and ensures uniformity in evaluation standards across different users and scenarios. Furthermore, this approach is particularly well-suited for environments where objective validation of product usage is critical, such as in clinical audits or quality assurance processes. Additionally, the centralized processing of images on the server allows for the integration of advanced image recognition algorithms and machine learning models, enabling future adaptations and improved accuracy over time.

Preferably, the accessibility of the selected medical product is determined based on inventory data received from an inventory management system arranged in communication with the server. By introducing a connection to an inventory management system, such a solution enables the server to directly access and evaluate inventory data, including for example current stock levels and availability of specific medical products. Specifically, real-time or regularly updated information allows for assessing whether the selected medical product is readily accessible within the healthcare unit, thereby ensuring that recommendations reflect actual stock conditions.

Advantageously, such an integration reduces reliance on manual stock-checking by healthcare personnel, improving efficiency and ensuring that stock-related issues are identified promptly. Proactively incorporating inventory data into the recommendation process minimizes treatment delays caused by unavailable medical products, supporting better continuity of care. Moreover, such a solution allows for seamless coordination with restocking systems, reducing the risk of critical shortages while optimizing inventory management practices across the healthcare unit.

Alternatively, the accessibility of the selected medical product is determined based on user input, via the electronic user device, indicating physical availability of the medical product at a storage location within the healthcare unit. Accordingly, by allowing healthcare personnel to manually input information about the availability of the selected medical product at a specific storage location using the electronic user device. Such an approach is particularly advantageous in environments where automated inventory management systems are unavailable or unreliable. By relying on user-reported data, the system can still assess accessibility and generate actionable recommendations, ensuring continuity of care. Additionally, such a solution supports real-time updates, enabling personnel to immediately indicate inventory changes, such as when items are restocked or relocated.

In an embodiment, generating the recommendation comprises determining, by the server, whether the product usage compliance level meets a predefined threshold and generating, by the server, application improvement instructions if the compliance level falls below the predefined threshold. Accordingly, by implementing a threshold-based evaluation at the server it may be possible to ensure that the product usage compliance level is systematically assessed against predefined standards, and similarly for each assessment. If the compliance level is found to be below the acceptable threshold, the server generates targeted application improvement instructions. Such instructions may then be used for guiding healthcare personnel on corrective measures, such as rearranging the medical product, ensuring proper adhesion, or correcting positioning, helping to standardize product usage practices.

Accordingly, the predefined thresholds preferably serve as an objective baseline for consistent evaluations, reducing variability in user assessments. Additionally, the improvement instructions offer clear, actionable feedback, enabling personnel of varying expertise levels to achieve optimal application outcomes. Such a process not only enhances patient care by preventing complications associated with poor medical product positioning but also streamlines workflows by reducing the likelihood of errors requiring follow-up interventions.

Preferably, the recommendation may also include a prioritized action based on the combined assessment of the accessibility and the product usage compliance level, wherein higher priority is assigned to actions addressing critical deficiencies. Such a prioritization mechanism may be used for ensuring that actions are ranked based on the combined assessment of medical product accessibility and usage compliance. Thus, by assigning higher priority to actions addressing critical deficiencies, such as severe non-compliance in product usage or significant stock shortages, the system ensures that the most urgent issues are addressed first. The prioritization may also enhance the efficiency of healthcare personnel by directing their attention to tasks with the greatest impact on patient safety and care quality.

In a possible embodiment of the present disclosure, the scheme further comprises tracking, by the server, compliance trends over time based on previously determined product usage compliance levels and using, by the server, the tracked trends to adapt the recommendations generated by the server. Accordingly, the server may be adapted to record compliance levels determined during previous assessments, creating a dataset that reflects the performance of personnel or units in applying medical products. Such trends may then be analyzed to identify recurring patterns, such as consistent deficiencies in adherence or alignment, and to predict potential compliance risks. The historical data may subsequently be used for dynamically adapting the recommendations, providing guidance tailored to address persistent or emerging issues specific to the healthcare unit or individual users.

By leveraging historical compliance data, it is possible to enable a shift from reactive to proactive management of product usage to ensure that systemic issues, such as recurring errors or gaps in training, are addressed comprehensively rather than on a case-by-case basis. The adaptive recommendations improve the relevance and precision of guidance provided to healthcare personnel, reducing the likelihood of repeated errors and enhancing overall application standards. Furthermore, the trend-tracking capability facilitates long-term quality assurance, providing healthcare administrators with valuable insights into compliance performance and areas needing improvement.

It may additionally be possible to store product usage compliance data and accessibility data in a database associated with the server, and as necessary generate periodic reports on medical product usage and product usage accuracy within the healthcare unit. Storing such data creates a centralized and organized repository that enables long-term tracking of medical product-related metrics. Such data points can include information on compliance trends, application errors, and inventory levels, providing a foundation for comprehensive analysis and informed decision-making. By applying such a data-driven approach to medical product management it is possible to ensure that facilities are equipped to address recurring deficiencies and optimize resource usage. For instance, reports can highlight areas where compliance consistently falls short or where inventory management practices need adjustment.

Preferably, the scheme according to the present disclosure further comprises calculating, by the server, a compliance-accessibility value based on a combination of the accessibility of the selected medical product within the healthcare unit and the determined product usage compliance level, tracking, by the server, the compliance-accessibility value over time, and using the tracked compliance-accessibility value to adapt the recommendations generated by the server. By introducing such an aggregated metric, it may be possible to form a comprehensive indicator of both stock availability and application accuracy, offering a unified measure to assess the overall effectiveness of medical product management. Tracking the compliance-accessibility value over time allows for the identification of trends, such as persistent deficiencies in availability or recurring compliance issues and enables dynamic adaptation of recommendations to address these challenges.

In an embodiment, the recommendation generated by the server includes a training module configured to provide instructions to the healthcare professional for improving the product usage compliance level. By incorporating a training module into the recommendation process, the system directly addresses gaps in user knowledge or technique that may contribute to non-compliance. Such a feature ensures that healthcare personnel receive targeted, actionable guidance tailored to the specific deficiencies identified during compliance evaluation and not only improves immediate product usage practices but also fosters skill development over time but contributes to higher overall standards of care and reducing the likelihood of repeated errors in clinical workflows.

In an embodiment it may be possible to configure the server to dynamically adjust predefined thresholds for the product usage compliance level and accessibility based on historical performance data and patient outcomes. As such, it may be possible to tailor the recommendations provided to reflect real-world performance and outcomes over time. Such an adaptability ensures that the thresholds remain clinically relevant, accounting for factors such as observed patient recovery patterns, variations in healthcare unit operations, or shifts in inventory usage trends.

Furthermore, it may be possible to evaluate by the server, one or more patient-specific parameters, wherein the patient-specific parameters include at least one of body mass index (BMI), mobility level, risk of skin breakdown, or length of hospitalization, and incorporating the evaluated parameters into the determination of the product usage compliance level. Incorporating patient-specific parameters into the determination of compliance levels allows the system to provide personalized assessments that account for the unique clinical conditions of each patient. For example, patients with limited mobility or a high risk of skin breakdown may require stricter adherence to product usage guidelines. Such an embodiment may thus be applied to ensure that recommendations are both relevant and aligned with individual patient needs, ultimately enhancing care quality and outcomes.

In a possible embodiment of the present disclosure, the accessibility of the selected medical product within the healthcare unit is dynamically updated based on inventory changes resulting from prior medical product usage or automated restocking, which in turn may be used to ensure that an up-to-date understanding of medical product accessibility is maintained by dynamically incorporating changes in inventory, whether from prior usage or automated restocking. Such real-time updates enable accurate recommendations that reflect the current stock availability, reducing the risk of delays caused by stockouts.

Possibly, the server may additionally be configured to categorize the selected medical product into at least one of a prophylactic dressing, a therapeutic dressing, or a monitoring dressing, wherein the categorization influences the determination of the product usage compliance level. It may as such be possible to align the compliance determination with the intended clinical purpose of the selected medical product. For instance, a prophylactic dressing may prioritize preventive adherence metrics, while a therapeutic dressing may require stricter evaluation of application integrity.

In a possible embodiment, the scheme according to the present disclosure further comprises generating, by the server, a notification to a healthcare professional if the product usage compliance level fails to meet a predefined threshold and the accessibility of a replacement medical product is insufficient to address the compliance issue. Generating a notification in response to both insufficient compliance and limited accessibility ensures that healthcare professionals are promptly alerted to critical deficiencies and helps mitigate risks associated with inadequate product usage or stock shortages by providing immediate guidance for corrective actions.

According to another aspect of the present disclosure, there is provided a computer system for managing medical product usage at a healthcare unit, comprising an electronic user device configured to receive input identifying a medical product selected for use in relation to a patient, and collect data from one or more input sources associated with the electronic user device, the data relating to the application of the selected medical product, a server arranged in communication with the electronic user device, the server configured to generate a recommendation for an action based on a combination of an accessibility of the selected medical product within the healthcare unit and a product usage compliance level determined based on the data collected using the electronic user device, and provide the recommendation to a healthcare professional associated with the healthcare unit. This aspect of the present disclosure provides similar advantages to those discussed above in relation to the previous aspects of the present disclosure.

According to a further aspect of the present disclosure, there is provided a computer program comprising instructions which, when executed by a computer or processor, cause a computer system to manage medical product usage at a healthcare unit, wherein the computer system comprises an electronic user device arranged in communication with a server, and wherein the computer program comprises code for receiving, at an electronic user device, input identifying a medical product selected for use on a patient, collecting, using the electronic user device, data from one or more input sources associated with the electronic user device, the data relating to the application of the selected medical product to the patient, determining, using a server arranged in communication with the electronic user device, a product usage compliance level for the selected medical product based on the data collected using the electronic user device, generating, by the server, a recommendation for an action based on a combination of an accessibility of the selected medical product within the healthcare unit and the determined product usage compliance level, and providing, by the server, the recommendation to a healthcare professional associated with the healthcare unit. Also this aspect of the present disclosure provides similar advantages to those discussed above in relation to the previous aspects of the present disclosure.

A software executed by the processing unit for operation in accordance to the present disclosure may be stored on a computer readable medium, being any type of memory device, including one of a removable non-volatile random access memory, a hard disk drive, a floppy disk, a CD-ROM, a DVD-ROM, a USB memory, an SD memory card, a solid state drive, other non-volatile flash based storage media, or a similar computer readable medium known in the art.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1 conceptually illustrates a computer system configured for managing medical product usage in a healthcare unit, in accordance with an embodiment of the present disclosure.

FIG. 2 schematically illustrates a healthcare professional using an electronic user device equipped with a camera to assess a medical product used in relation to a patient, in accordance with an embodiment of the present disclosure.

FIG. 3 schematically illustrates the interaction between the electronic user device, a server, and an inventory management system for assessing product usage compliance and accessibility.

FIG. 4 is a flowchart illustrating the steps of performing the method for managing medical product usage, in accordance with an embodiment of the present disclosure.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein, rather, these embodiments are provided for thoroughness and completeness, and fully convey the scope of the present disclosure to the skilled person. Like reference characters refer to like elements throughout.

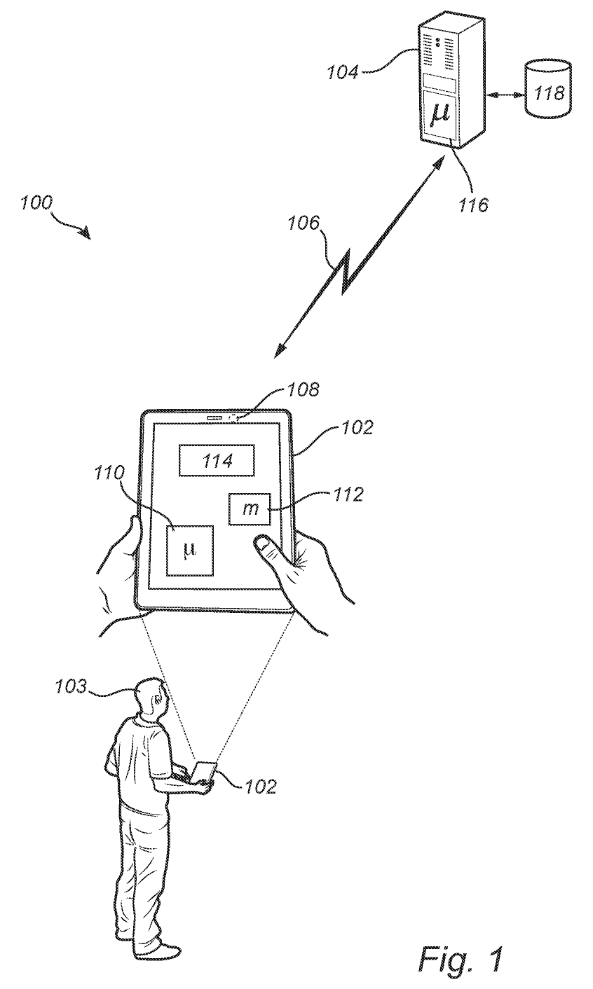

Turning now to the drawings and to FIG. 1 in particular, there is conceptually illustrated a computer system 100 configured for managing medical product usage in a healthcare unit, in accordance with an embodiment of the present disclosure. The computer system 100 comprises an electronic user device 102 and a server 104, which are arranged in communication via a network connection 106.

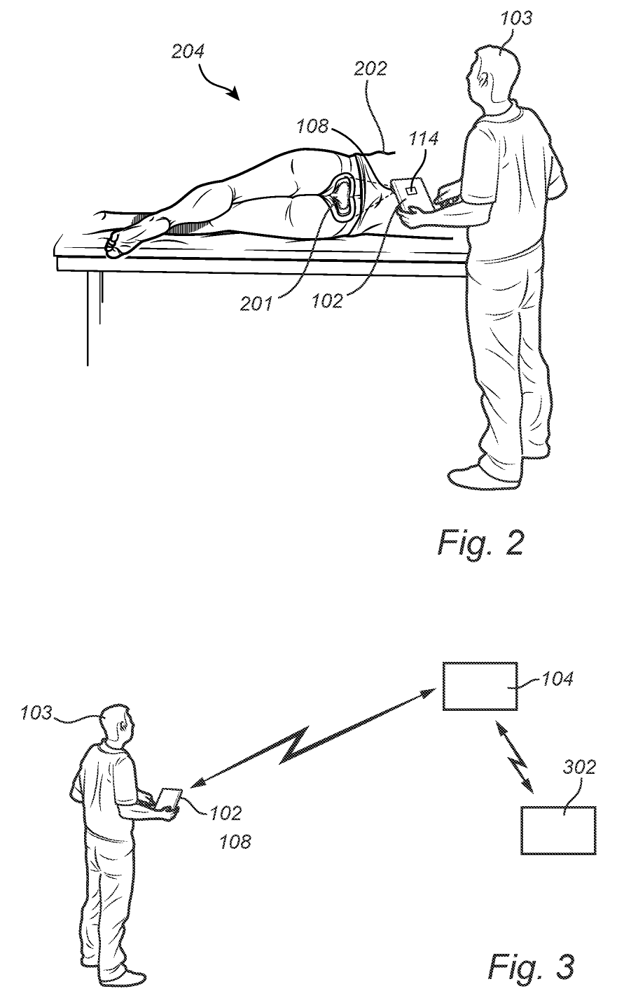

The electronic user device 102 is operated by healthcare personnel 103 and includes a camera 108, a processing unit 110, a memory 112, and a graphical user interface (GUI) 114. The camera 108 is used to capture images of medical product, here in the form of a wound dressing applied to a patient (as will be further illustrated in FIG. 2), providing visual data that can be analyzed to determine product usage compliance. The GUI 114 facilitates interaction between the healthcare personnel 103 and the server 104 allowing the healthcare personnel 103 to input information about the wound dressing or view recommendations received from the server 104. The processing unit 110 processes the collected data locally as needed, such as formatting image data for transmission to the server 104. The memory 112 is configured to store captured data temporarily or persistently, ensuring continuity in case of network delays or interruptions.

The server 104 includes its own processing unit 116 and memory 118, enabling it to perform advanced computational tasks, including analyzing the captured image data to assess compliance metrics, integrating inventory data from an associated inventory management system, and generating actionable recommendations for the healthcare professional. The processing capabilities of the server 104 allow the server 104 to handle large volumes of data and provide dynamic recommendations tailored to specific patient and healthcare unit conditions.

The network connection 106, for example being an Internet connection, ensures seamless communication between the user device and the server, supporting real-time data exchange. For example, image data captured by the camera 108 is transmitted to the server 104 for analysis, and the resulting compliance assessment, combined with inventory accessibility information, is returned to the user device 102 for display on the GUI 114.

For reference, the processing units 110, 116 may be implemented as general-purpose processors, application-specific processors, or distributed processing components. These may include hardware such as field-programmable gate arrays (FPGAs), system-on-chip (SOC) designs, or a combination of hardware and software components. The memories 112, 118 may consist of volatile or non-volatile storage, capable of retaining data structures, object code, or script components necessary for executing the processes described herein.

The computer system 100 forms the backbone of the disclosed method, enabling the collection, analysis, and dissemination of data critical for managing medical product usage effectively. Additionally, the integration of the camera 108 with the system 100 allows for enhanced compliance assessments, leveraging visual data to identify application errors and provide targeted recommendations for improvement.

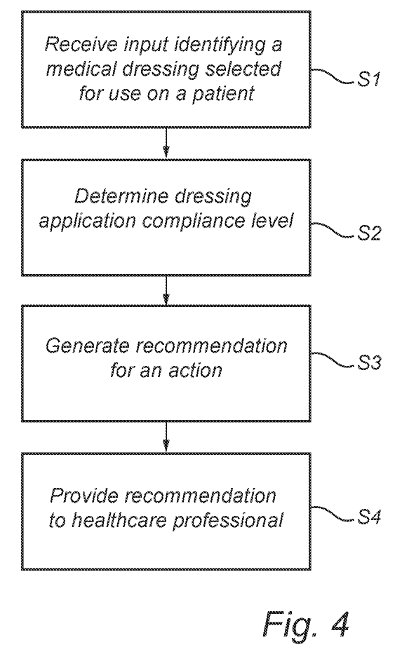

FIG. 2 schematically illustrated, with further reference to FIG. 4, a healthcare professional 103 operating the electronic user device 102 for assessing a wound dressing 201 applied to a patient 202 in a healthcare unit 204. The interaction begins when the healthcare professional 103 initiates the process to manage the medical product usage for the patient 202, where the patient 202 generally is associated with the health care unit 204 (such as e.g. currently receiving treatment at the healthcare unit 204).

As a first step, input is received, S1, at the electronic user device 102 to identify the medical product selected for use. Such an input may be entered manually via a graphical user interface GUI 114 or pre-filled based on existing patient records or inventory data. The healthcare professional 103 can then proceed to verify the application of the selected dressing on the patient 202.

To determine, S2, the product usage compliance level, the system 100 may be configured to offer multiple data collection pathways. The camera 108 of the user device 102 may be used to capture images of the medical product, such as the wound dressing 201. As is exemplary illustrated in FIG. 2, the wound dressing 201 is a prophylactic dressing aimed at preventing pressure ulcers, where the wound dressing has previously been applied to the lower back of the patient 202. The camera 108 is here used for capturing an image of the wound dressing 201 for arranging the server 104 to assess if the application of the wound dressing 201 is fulfilling predefined criteria, to determine a product usage compliance level. Alternatively, the product usage compliance level may be determined through structured questions presented on the GUI 114, such as whether the wound dressing fully adheres to the skin or if its placement aligns with the intended site. Such a flexibility ensures that compliance can be reliably assessed even when imaging is not practical.

In parallel, the system 100 assesses the accessibility of the selected wound dressing 201 within the healthcare unit 204. This information is typically retrieved automatically from an inventory management system (as presented in FIG. 3) connected to the server 104 using e.g. a network connection. However, in environments lacking automation, accessibility data may instead be gathered manually by the healthcare personnel 103 by physically inspecting a storage room and inputting the findings into the GUI 114. The suggested dual capability ensures the system 100 remains functional across diverse healthcare settings.

Based on the combination of the product usage compliance level and accessibility data, the server 104 generates, S3, a recommendation. The generated recommendation may include actionable guidance such as corrective measures to improve product usage, or alerts indicating the need to restock the dressing due to low inventory levels. The recommendation may in line with the present disclosure be tailored to the current situation and dynamically adapt based on historical trends and patient-specific parameters, ensuring relevance and precision.

Finally, the recommendation is provided, S4, to the electronic user device 102 and presented to the healthcare professional 103 via the GUI 114. Such a functionality allows the healthcare professional 103 to take immediate corrective actions, such as reapplying the wound dressing 201 to improve compliance or initiating a restocking request if inventory levels are insufficient. The seamless interaction between the user device 102, the healthcare professional 103, and the patient 202 ensures that medical dressings 201 are used effectively, supporting both patient safety and operational efficiency within the healthcare unit 204.

By integrating flexible data acquisition methods and real-time analysis capabilities, the system 100 empowers healthcare personnel 103 to address both compliance and accessibility challenges proactively, thereby enhancing the overall standard of wound care management.

Turning finally to FIG. 3, there is schematically illustrated the interaction between the electronic user device 102, the server 104, and an optional inventory management system 302, demonstrating how information flows between components to manage medical product usage within a healthcare unit.

The electronic user device 102, operated by healthcare personnel, initiates the process by collecting patient-specific data. The data may for example include inputs identifying the selected medical dressing 201 and information relating to the application compliance level for the wound dressing 201. Data collection methods may involve images captured by the camera 108 or responses entered via the graphical user interface GUI 114. These inputs are transmitted in real time to the server 104 via the (preferably secure) network connection 106. The server 104 may be implemented as a cloud-based server, providing centralized, scalable data processing capabilities, or as an on-premises system integrated into the infrastructure of the health care unit 204.

Once the server 104 receives the data, it performs real-time analysis to determine the product usage compliance level. The process may for example involve computational tasks such as image analysis using pattern recognition algorithms or processing questionnaire responses to assess alignment, adhesion, and other application metrics. Simultaneously, the server 104 queries the inventory management system 300, retrieving accessibility data for the selected medical dressing. The inventory system tracks stock levels and restocking schedules, ensuring that the server has up-to-date information about dressing availability within the healthcare unit.

The server 104 combines the analyzed compliance data with the accessibility information to generate a tailored recommendation. The recommendation may as such be seen as being derived from predefined rules or thresholds stored on the server 104 and may involve further processing based on historical trends, patient-specific parameters, or organizational policies. For example, if the compliance level is below a predefined threshold and the stock level is insufficient, the server 104 may prioritize corrective actions, such as generating training instructions or initiating restocking requests. The recommendation generation process involves real-time data processing and decision-making logic, ensuring that the output is relevant and actionable.

The recommendation is then generally transmitted back to the electronic user device 102 for immediate presentation to the healthcare professional 103 via the GUI 114. Such an implementation may allow the healthcare personnel 103 to address identified issues promptly, such as improving product usage or requesting additional supplies. The system 100 further ensures that all data exchanges occur in real time or near real time, minimizing delays in the decision-making process and reducing risks associated with improper product usage or insufficient inventory.

Furthermore, the control functionality of the present disclosure may be implemented using existing computer processors, or by a special purpose computer processor for an appropriate system, incorporated for this or another purpose, or by a hardwire system. Embodiments within the scope of the present disclosure include program products comprising machine-readable medium for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, solid state drives or other non-volatile flash based storage devices, or any other medium which can be used to carry or store desired program code in the form of machine-executable instructions or data structures and which can be accessed by a general purpose or special purpose computer or other machine with a processor. When information is transferred or provided over a network or another communications connection either hardwired, wireless, or a combination of hardwired or wireless to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures may show a sequence the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the present disclosure has been described with reference to specific exemplifying embodiments thereof, many different alterations, modifications and the like will become apparent for those skilled in the art.

In addition, variations to the disclosed embodiments can be understood and effected by the skilled addressee in practicing the claimed present disclosure, from a study of the drawings, the disclosure, and the appended claims. Furthermore, in the claims, the word ”comprising” does not exclude other elements or steps, and the indefinite article ”a” or ”an” does not exclude a plurality.

1. A method for managing medical product usage at a healthcare unit, comprising:
receiving, at an electronic user device, input identifying a medical product selected for use in relation to a patient,
determining, using the electronic user device and based on data obtained from one or more input sources associated with the electronic user device, a product usage compliance level for the selected medical product ,
generating, by a server arranged in communication with the electronic user device, a recommendation for an action based on a combination of an accessibility of the selected medical product within the healthcare unit and the determined product usage compliance level, and
providing, by the server, the recommendation to a healthcare professional associated with the healthcare unit.

2. The method of claim 1, wherein determining the product usage compliance level comprises:
presenting, at a graphical user interface (GUI) configured at the electronic user device, a set of product usage-related questions, and
receiving, at the GUI of the electronic user device, responses from a user, wherein the responses are used to determine the product usage compliance level.

3. The method of claim 1, wherein determining the product usage compliance level comprises:
capturing, using an image device associated with the electronic user device, an image of the used medical product, and
analyzing, using the server, the image to determine the product usage compliance level.

4. The method of claim 1, wherein the accessibility of the selected medical product is determined based on inventory data received from an inventory management system arranged in communication with the server.

5. The method of claim 1, wherein the accessibility of the selected medical product is determined based on user input, via the electronic user device, indicating physical availability of the medical product at a storage location within the healthcare unit.

6. The method of claim 1, wherein generating the recommendation comprises:
determining, the server, whether the product usage compliance level meets a predefined threshold, and
generating, by the server, application improvement instructions if the compliance level falls below the predefined threshold.

7. The method of claim 1, wherein generating the recommendation comprises:
generating, by the server, a prompt to restock the selected medical product if the accessibility falls below a predefined threshold.

8. The method of claim 1, wherein the recommendation includes a prioritized action based on the combined assessment of the accessibility and the product usage compliance level, wherein higher priority is assigned to actions addressing critical deficiencies.

9. The method of claim 1, further comprising:
tracking, by the server, compliance trends over time based on previously determined product usage compliance levels, and
using, by the server, the tracked trends to adapt the recommendations generated by the server.

10. The method of claim 1, further comprising:
storing, using the server, product usage compliance data and accessibility data in a database associated with the server.

11. The method according to claim 10, further comprising:
generating, using the server, periodic reports on medical product usage and product usage accuracy within the healthcare unit.

12. The method of claim 1, further comprising:
calculating, by the server, a compliance-accessibility value based on a combination of the accessibility of the selected medical product within the healthcare unit and the determined product usage compliance level,
tracking, by the server, the compliance-accessibility value over time, and
using the tracked compliance-accessibility value to adapt the recommendations generated by the server.

13. The method of claim 1, wherein the recommendation generated by the server includes a training module configured to provide instructions to the healthcare professional for improving the product usage compliance level.

14. The method of claim 1, further comprising:
generating, by the server, an aggregated compliance score for a plurality of patients based on tracked product usage compliance levels across multiple patients within the healthcare unit.

15. The method of claim 1, further comprising:
evaluating, by the server, one or more patient-specific parameters, wherein the patient-specific parameters include at least one of: body mass index (BMI), mobility level, risk of skin breakdown, or length of hospitalization, and incorporating the evaluated parameters into the determination of the product usage compliance level.

16. The method of claim 1, further comprising:
categorizing, by the server, the selected medical product into at least one of a prophylactic dressing, a therapeutic dressing, or a monitoring dressing, wherein the categorization influences the determination of the product usage compliance level.

17. The method of claim 1, further comprising:
generating, by the server, a notification to a healthcare professional if the product usage compliance level fails to meet a predefined threshold and the accessibility of a replacement medical product is insufficient to address the compliance issue.

18. A computer system for managing medical product usage at a healthcare unit, comprising:
an electronic user device configured to:
receive input identifying a medical product selected for use in relation to a patient, and
collect data from one or more input sources associated with the electronic user device, the data relating to the application of the selected medical product,
a server arranged in communication with the electronic user device, the server configured to:
generate a recommendation for an action based on a combination of an accessibility of the selected medical product within the healthcare unit and a product usage compliance level determined based on the data collected using the electronic user device, and
provide the recommendation to a healthcare professional associated with the healthcare unit.

19. The system of claim 18, wherein the electronic user device comprises an image capture device configured to capture an image of the used medical product, and the server is configured to analyze the image to determine the product usage compliance level.

20. A computer program comprising instructions which, when executed by a computer or processor, cause a computer system to manage medical product usage at a healthcare unit, wherein the computer system comprises an electronic user device arranged in communication with a server, and wherein the computer program comprises:
code for receiving, at an electronic user device, input identifying a medical product selected for use in relation to a patient,
collecting, using the electronic user device, data from one or more input sources associated with the electronic user device, the data relating to the application of the selected medical product,
determining, using a server arranged in communication with the electronic user device, a product usage compliance level for the selected medical product based on the data collected using the electronic user device,
generating, by the server, a recommendation for an action based on a combination of an accessibility of the selected medical product within the healthcare unit and the determined product usage compliance level, and
providing, by the server, the recommendation to a healthcare professional associated with the healthcare unit.
